(19) United States
(12) Patent Application Publication
(10) Pub. No.: US 2025/0019764 A1
(43) Pub. Date: Jan. 16, 2025

(54) METHODS FOR THE PROGNOSIS AND TREATMENT OF TEMPORAL LOBE EPILEPSY

(71) Applicant: ARIZONA BOARD OF REGENTS ON BEHALF OF THE UNIVERSITY OF ARIZONA, Tucson, AZ (US)

(72) Inventor: Martin Weinand, Tucson, AZ (US)

(21) Appl. No.: 18/754,715

(22) Filed: Jun. 26, 2024

Related U.S. Application Data
(60) Provisional application No. 63/512,791, filed on Jul. 10, 2023.

(51) Int. Cl. C12Q 1/6883

(52) U.S. Cl. CPC C12Q 1/6883; C12Q 2600/112; C12Q 2600/158

(57) ABSTRACT

Differential expression of specific leukocyte genes involved in canonical pathways of neuroinflammation, oxidative stress, and lipid peroxidation, GABA (γ-aminobutyric acid) inhibition, and AMPA and NMDA receptor signaling is able to predict the severity of temporal lobe epilepsy (TLE). For example, high seizure frequency-TLE is distinguished prognostically from low seizure frequency-TLE by differentially increased specific leukocyte gene expression involved in GABA inhibition and NMDA receptor signaling. Thus, high and low seizure frequency patients represent two mechanistically different forms of temporal lobe epilepsy based on leukocyte gene expression and potentially require distinct treatments.

### CROSS-REFERENCE TO RELATED APPLICATIONS

This application is a non-provisional and claims benefit of U.S. Provisional Application No. 63/512,791 filed Jul. 10, 2023, the specification of which is incorporated herein in their entirety by reference.

### STATEMENT REGARDING FEDERALLY SPONSORED RESEARCH OR DEVELOPMENT

This invention was made with government support under Grant No. MH065151 awarded by National Institutes of Health. The government has certain rights in the invention.

### FIELD OF THE INVENTION

The present invention features methods for the prognosis and treatment of temporal lobe epilepsy (TLE); specifically, differential leukocyte gene expression predicts human temporal lobe epilepsy seizure frequency.

### BACKGROUND OF THE INVENTION

Temporal lobe epilepsy (TLE) is the most common and medically refractory form of focal epilepsy in adults. Among patients with TLE, approximately two-thirds may be rendered seizure-free with anticonvulsant therapy. For those patients in whom seizures persist despite maximum medical management, epilepsy surgery is a potentially curative treatment. Surgical treatment of TLE may include ablation or resection of temporal lobe epileptic tissue, including stereotactic laser amygdalohippocampotomy (SLAH) or amygdalohippocampectomy (AH) with or without anterior temporal lobectomy (ATL), respectively. In patients with medically refractory TLE, seizure freedom may occur in approximately 60% of patients treated with ablative surgery (i.e., SLAH) and up to 80% of patients treated with respective surgery (i.e., AH with or without ATL).

Currently, medical management of intractable TLE is based on the clinical diagnosis of intractable TLE (i.e., not based on genetic or biological pathway pathophysiology). A more tailored approach for TLE treatment, based on mechanistically separate categories of disease of high and low seizure frequency, will allow more personalized pharmacologic therapy directed at TLE genomic and biological pathway-specific pathophysiology for the high and low seizure frequency categories of disease. Thus, the present invention features a more personalized genomic-based therapy to improve temporal lobe epilepsy, including reduced seizure frequency.

### BRIEF SUMMARY OF THE INVENTION

It is an objective of the present invention to provide methods that allow for the prognosis and treatment of temporal lobe epilepsy (TLE); specifically, differential leukocyte gene expression predicts human temporal lobe epilepsy seizure frequency, as specified in the independent claims. Embodiments of the invention are given in the dependent claims. Embodiments of the present invention can be freely combined with each other if they are not mutually exclusive.

Human TLE appears to be a heterogeneous disease, with dichotomous functional neuroanatomic, microcellular, and signaling pathways based on seizure frequency. For example, TLE patients with high seizure frequency have increased strength of hippocampal to parahippocampal functional connectivity, whereas TLE patients with low seizure frequency possess increased functional connectivity between the amygdala and parahippocampus. In addition, greater seizure frequency in human TLE is associated with a trend towards increased neuronal loss measured by decreased N-acetyl-aspartate (NAA). Furthermore, in high seizure frequency TLE, compared to low seizure frequency TLE, altered signaling pathways appear to be more predominately deactivated. Finally, low seizure frequency TLE is maintained by increased expression of genes involved in synaptic plasticity. Thus, divergent patterns of temporal lobe functional connectivity, neuronal density, and gene expression might differentiate human TLE based on high and low seizure frequency. A biomarker dichotomizing TLE based on a clinical measure of epileptogenicity could have implications for fundamental pathophysiological and successful therapeutic strategy differences between low and high seizure frequency.

In some embodiments, the present invention features a method of predicting human temporal lobe epilepsy (TLE) seizure frequency in a subject in need thereof. The method may comprise obtaining a biological sample (e.g., a blood sample) from the subject and measuring the expression levels of one or more leukocyte genes in the biological sample (e.g., the blood sample) from the subject. In some embodiments, the subject is predicted to have severe TLE if the levels of the one or more leukocyte genes are down-regulated, and the subject is predicted to have mild TLE if the levels of the one or more leukocyte genes are upregulated.

In other embodiments, the present invention features a method of predicting human temporal lobe epilepsy (TLE) seizure frequency in a subject in need thereof. The method may comprise obtaining a biological sample (e.g., a blood sample) from the subject and measuring the expression levels of one or more leukocyte genes in the biological sample (e.g., the blood sample) from the subject. In some embodiments, the subject is predicted to have severe TLE if the levels of the one or more leukocyte genes are down-regulated, and the subject is predicted to have mild TLE if the levels of the one or more leukocyte genes are upregulated.

In further embodiments, the present invention features a method of treating a subject with temporal lobe epilepsy (TLE). First, the method comprises determining the severity of the TLE in a subject in need thereof by obtaining a biological sample (e.g., a blood sample) from the subject and measuring the expression levels of one or more leukocyte genes in the biological sample (e.g., the blood sample) from the subject. If the expression levels of the one or more leukocyte genes are down-regulated, the subject has severe TLE, and if the expression levels of the one or more leukocyte genes are upregulated, the subject has mild TLE. Second, the method comprises administering a treatment to the subject based on whether the subject has severe TLE or mild TLE.

One of the unique and inventive technical features of the present invention is the identification of biomarkers (e.g., leukocyte biomarkers). Without wishing to limit the invention to any theory or mechanism, it is believed that the technical feature of the present invention advantageously provides for methods to predict the severity of temporal lobe epilepsy (TLE). None of the presently known prior references or work has the unique inventive technical feature of the present invention.

Furthermore, the prior references teach away from the present invention. For example, seizure frequency has traditionally served as a clinical measure of temporal lobe epilepsy severity (i.e., epileptogenicity) and as a quantitative measure of the TLE response to medical and surgical therapy.

However, in the emerging era of personalized medicine, the development of biomarkers may improve the quantitative assessment of the severity and response to treatment of various disease states. Biomarkers of the disease may offer insight into disease pathophysiology, potentially improving the development of novel therapies. Biomarkers may predict the response of disease to medical or surgical treatment. As described herein, one such biomarker of disease is leukocyte gene expression. Leukocyte gene expression may reflect central nervous system (CNS) disease severity, possibly through brain leukocyte trafficking. Systemic leukocyte gene expression may recapitulate the pathophysiology of CNS disease, indicate disease diagnosis and severity, and predict response to therapy. For example, the down-regulation of leukocyte genes for mitochondrial protein synthesis, mitophagy, and stress defense is associated with the diagnosis of Parkinson's Disease. Leukocyte gene expression for B-cell receptor signaling and apoptosis correlates with the severity of depression. In refractory temporal lobe epilepsy, leukocyte gene expression for lipid metabolism, oligodendrocyte morphology, inflammatory response, and astrocyte development has predictive value for seizure-freedom response to stereotactic laser amygdalohippocampotomy (SLAH).

Furthermore, the inventive technical features of the present invention contributed to a surprising result. For example, the Inventors surprisingly found that high seizure frequency TLE is distinguishable from low seizure frequency TLE by increased leukocyte gene expression related to GABA (γ-aminobutyric acid) inhibition and NMDA (NMethyl-d-aspartate) receptor signaling. They also found consistent patterns of suppressed or enhanced processes involving phagosome formation, CREB signaling, and autophagy in the leukocytes of high and low seizure frequency patients. These findings suggest that there are mechanistically two distinct forms of TLE based on leukocyte gene expression.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a multidimensional scaling plot (MDS) generated using edgeR showing the segregation of the low and high seizure frequency patients' leukocyte transcriptional profile. The three lowest and three highest seizure frequency samples were used to generate the plot. Numbered sample IDs indicate patients from the list in Table 1.

FIG. 2 shows heatmap generated in edgeR using the 13 most variable genes across samples. Unsupervised clustering showing the grouping of low (#5, #3, #2) and high (#6, #15, #11) seizure frequency patients. X-axis indicates sample IDs from subjects listed in Table 1. Red indicates a higher level of comparative expression while blue indicates a lower level of expression.

FIG. 3 shows leukocyte differentially expressed genes (DEGs) involved in neuroinflammation are upregulated in low but downregulated in high temporal lobe epilepsy seizure frequency. Figure prepared with BioRender.com

FIG. 4 shows leukocyte differentially expressed genes (DEGs) involved in oxidative stress, and lipid peroxidation are upregulated in low but downregulated in high temporal lobe epilepsy seizure frequency. Figure prepared with BioRender.com

FIG. 5 shows leukocyte differentially expressed genes (DEGs) involved in glutamate/GABA-mediated excitotoxicity in temporal lobe epileptogenicity, which are upregulated in low but downregulated in high temporal lobe epilepsy seizure frequency. Figure prepared with BioRender.com

FIG. 6 shows leukocyte differentially expressed genes (DEGs) involved in NMDA facilitation and GABA inhibition which are upregulated in high compared to low temporal lobe epilepsy seizure frequency patients. Figure prepared with BioRender.com

FIG. 7 shows upstream regulators predicted to drive the temporal lobe epilepsy response in leukocytes with an overlap p-value <0.05. The NFKB complex is a key node in this network. Figure prepared using Ingenuity Pathway Analysis (IPA).

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. The term does not denote a particular age or sex. Thus, adult and newborn subjects, as well as fetuses, whether male or female, are intended to be included. In one embodiment, the subject is a mammal (e.g., a human) having a disease, disorder, or condition described herein. In another embodiment, the subject is a mammal (e.g., a human) at risk of developing a disease, disorder, or condition described herein. A “patient” is a subject afflicted with a disease or disorder. The term “patient” includes human and veterinary subjects. In certain instances, the term patient refers to a human.

The terms “treating” or “treatment” refer to any indicia of success or amelioration of the progression, severity, and/or duration of a disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread, or worsening of a disease or disorder or of one or more symptoms thereof. In certain cases, the beneficial effects that a subject derives from a prophylactic or therapeutic agent do not result in a cure of the disease or disorder.

The term “effective amount” as used herein refers to the amount of a therapy which is sufficient to reduce and/or ameliorate the severity and/or duration of a given disease, disorder, or condition and/or a symptom related thereto. This term also encompasses an amount necessary for the reduction or amelioration of the advancement or progression of a given disease, disorder, or condition, reduction or amelioration of the recurrence, development or onset of a given disease, disorder, or condition, and/or to improve or enhance the prophylactic or therapeutic effect(s) of another therapy. In some embodiments, “effective amount,” as used herein, also refers to the amount of therapy provided herein to achieve a specified result.

As used herein, and unless otherwise specified, the term “therapeutically effective amount” is an amount sufficient to provide a therapeutic benefit in the treatment or management of a disease or to delay or minimize one or more symptoms associated with a disease. A therapeutically effective amount of calcimycin means an amount of therapeutic agent, alone or in combination with other therapies, which provides a therapeutic benefit in the treatment or management of a disease. The term “therapeutically effective amount” can encompass an amount that improves overall therapy, reduces or avoids symptoms or causes, or enhances the therapeutic efficacy of another therapeutic agent.

The terms “administering” and “administration” refer to methods of providing a pharmaceutical preparation to a subject. Such methods are well known to those skilled in the art and include, but are not limited to, administering the compositions intranasally, parenterally (e.g., intravenously and subcutaneously), by intramuscular injection, by intraperitoneal injection, intrathecally, transdermally, extracorporeally, topically, orally or the like.

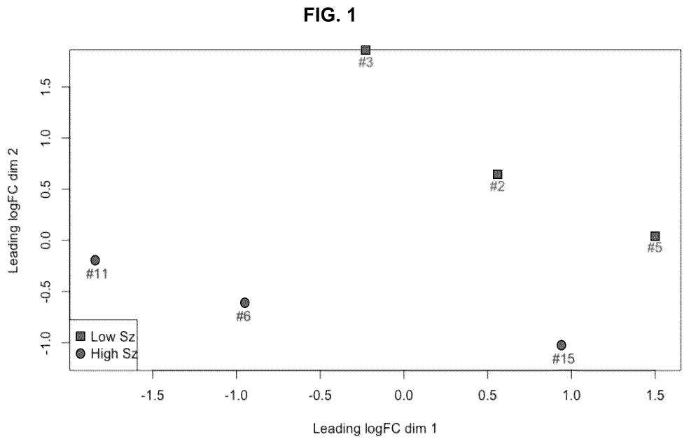

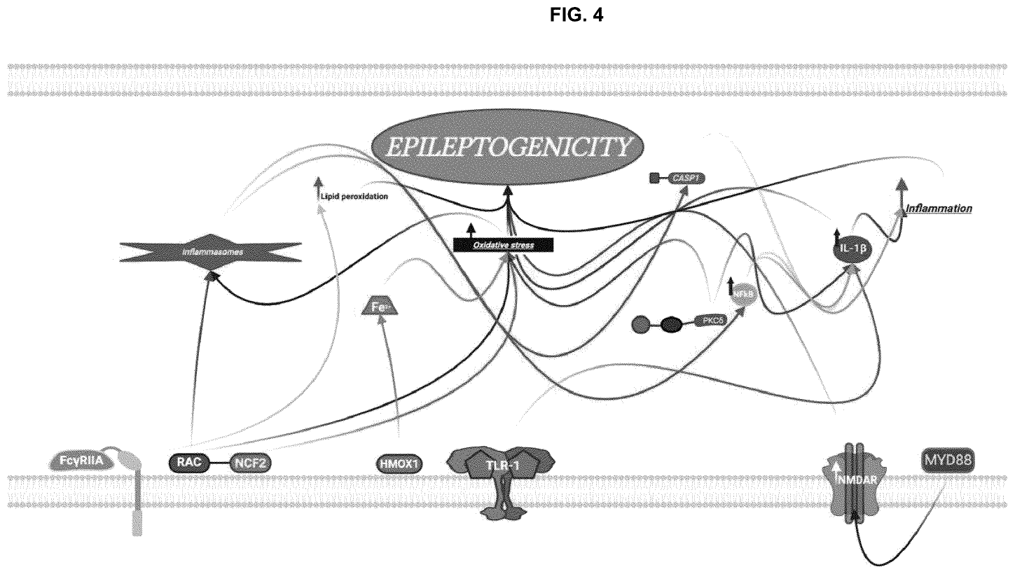

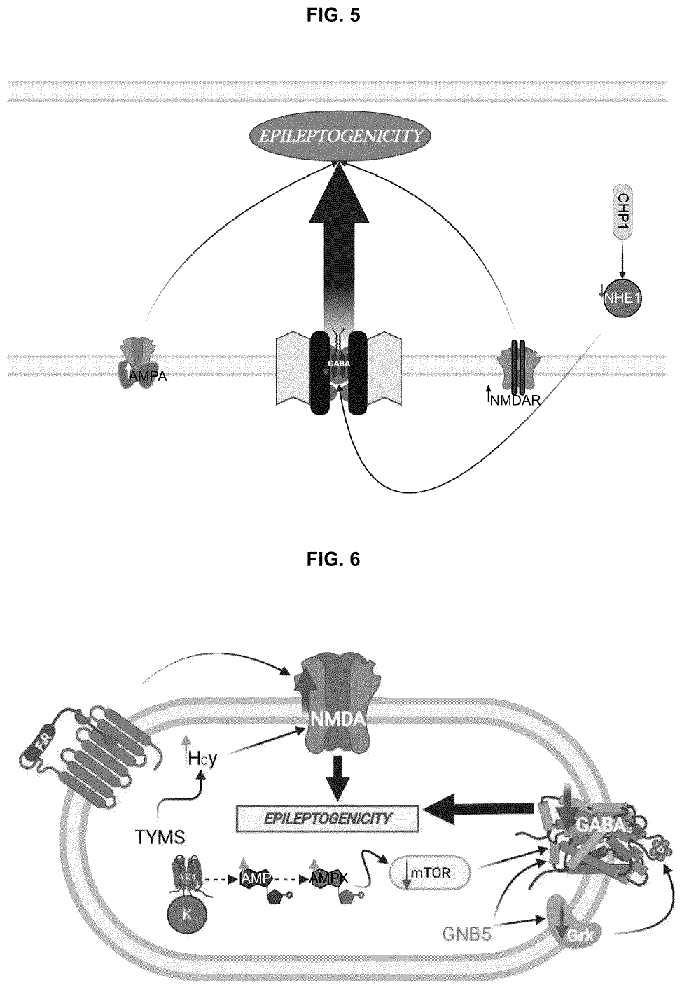

Referring now to FIGS. 1-7, the present invention features methods for the prognosis and treatment of temporal lobe epilepsy (TLE); specifically, differential leukocyte gene expression predicts human temporal lobe epilepsy seizure frequency.

The present invention features a method of predicting human temporal lobe epilepsy (TLE) seizure frequency in a subject in need thereof. In some embodiments, the method comprises obtaining or having obtained a biological sample from the subject and measuring the expression levels of one or more leukocyte genes in the biological sample from the subject. In some embodiments, the subject is predicted to have severe TLE if the expression levels of one or more leukocyte genes are down-regulated, or the subject is predicted to have mild TLE if the levels of the one or more leukocyte genes are upregulated. For example, if the expression level one or more leukocyte genes selected from a group consisting (or consisting essentially) of Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), and Caspase-1 (CASP1) are upregulated then the subject is predicted to have mild TLE. In other embodiments, the subject is predicted to have severe TLE if the expression levels of one or more leukocyte genes are up-regulated, or the subject is predicted to have mild TLE if the levels of one or more leukocyte genes are down-regulated. For example, if the expression level one or more leukocyte genes selected from a group consisting (or consisting essentially) of thymidylate synthase (TYMS), adenylate kinase 1 (AK1), coagulation factor II thrombin receptor (F2R), and guanine nucleotide-binding protein subunit beta-5 (GNB5) are up-regulated, the subject is predicted to have severe TLE.

In some embodiments, the biological sample is a blood sample. In some embodiments, the biological sample is a brain sample. In some embodiments, the biological sample is a skin sample. In some embodiments, the biological sample is spinal fluid. In some embodiments, the biological sample is a cheek swab.

In other embodiments, the method may comprise obtaining or having obtained a blood sample from the subject and measuring the expression levels of one or more leukocyte genes in the blood sample from the subject. In some embodiments, the subject is predicted to have severe TLE if the levels of the one or more leukocyte genes are down-regulated, and the subject is predicted to have mild TLE if the levels of the one or more leukocyte genes are upregulated.

In some embodiments, the method may comprise obtaining or having obtained a blood sample from the subject; and measuring the expression levels of five or more leukocyte genes in the blood sample from the subject. In other embodiments, the method may comprise obtaining or having obtained a blood sample from the subject; and measuring the expression levels of ten or more leukocyte genes in the blood sample from the subject. In some embodiments, the subject is predicted to have severe TLE if (i) the expression levels of the leukocyte genes selected from a group consisting (or consisting essentially) of Thymidylate Synthase (TYMS), Adenylate Kinase 1 (AK1), Coagulation Factor II Thrombin Receptor (F2R), and G Protein Subunit Beta 5 (GNB5) are upregulated and/or (ii) the expression level of the leukocyte genes selected from a group consisting (or consisting essentially) of Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), and Caspase-1 (CASP1) are down-regulated. In some embodiments, the subject is predicted to have mild TLE if (i) the expression levels of the leukocyte genes selected from a group consisting (or consisting essentially) of NCF2, HMOX1, FCGR2A, PRKCD, CHP1, IFNGR1, RHOB, RAC2, TLR1, TNFRSF1A, LYN, MYD88, and CASP are upregulated and/or (ii) the expression levels of the leukocyte genes selected from a group consisting (or consisting essentially) of TYMS, AK1, F2R, and GNB5 are downregulated.

The present invention may further feature a method of treating a subject with temporal lobe epilepsy (TLE). First, the method comprises determining the severity of the TLE in a subject in need thereof as described herein. For example, the method may comprise obtaining or having obtained a biological sample (e.g., a blood sample) from the subject and measuring the expression levels of one or more leukocyte genes in the biological sample (e.g., the blood sample) from the subject. If the expression levels of one or more leukocyte genes are down-regulated, the subject has severe TLE, and if the expression levels of the one or more leukocyte genes are upregulated, the subject has mild TLE. Second, the method comprises administering a treatment to the subject based on whether the subject has severe TLE or mild TLE.

In other embodiments, the method may comprise determining the severity of the TLE in a subject in need thereof by obtaining or having obtained a biological sample (e.g., a blood sample) from the subject and measuring the expression levels of ten or more leukocyte genes in the blood sample from the subject. In some embodiments, the subject is predicted to have severe TLE if (i) the expression levels of the leukocyte genes selected from a group consisting of TYMS, AK1, F2R, and GNB5 are upregulated and/or (ii) the expression level of the leukocyte genes selected from a group consisting of NCF2, HMOX1, FCGR2A, PRKCD, CHP1, IFNGR1, RHOB, RAC2, TLR1, TNFRSF1A, LYN, MYD88, and CASP are down-regulated; and the subject is predicted to have mild TLE if (i) the expression level of the leukocyte genes selected from a group consisting of NCF2, HMOX1, FCGR2A, PRKCD, CHP1, IFNGR1, RHOB, RAC2, TLR1, TNFRSF1A, LYN, MYD88, and CASP are upregulated and/or (ii) the expression levels of the leukocyte genes selected from a group consisting of TYMS, AK1, F2R, and GNB5 are downregulated. In some embodiments, the method further comprises administering a treatment to the subject based on whether the subject has severe TLE or mild TLE.

The present invention may also feature a method for determining the effectiveness of a treatment for mild temporal lobe epilepsy (TLE) in a subject in need thereof. The method may comprise obtaining a first biological sample (e.g., a first blood sample) from the subject, measuring the expression levels of one or more leukocyte genes in the first biological sample (e.g., the first blood sample) from the subject, administering a treatment to the subject, obtaining a second biological sample (e.g., a second blood sample) from the subject, and measuring the expression levels of one or more leukocyte genes in the second biological sample (e.g., the second blood sample) from the subject. In some embodiments, the treatment is effective if (i) the expression levels of the one or more leukocyte genes selected from a group consisting (or consisting essentially) of NCF2, HMOX1, FCGR2A, PRKCD, CHP1, IFNGR1, RHOB, RAC2, TLR1, TNFRSF1A, LYN, MYD88, and CASP are down-regulated and/or (ii) the expression levels of the leukocyte genes selected from a group consisting of TYMS, AK1, F2R, and GNB5 are upregulated when comparing the first biological sample (e.g, the first blood sample) to the second biological sample (e.g., the second blood sample). In some embodiments, the treatment is ineffective if the expression levels of one or more leukocyte genes selected from a group consisting (or consisting essentially) of NCF2, HMOX1, FCGR2A, PRKCD, CHP1, IFNGR1, RHOB, RAC2, TLR1, TNFRSF1A, LYN, MYD88, and CASP are upregulated or unchanged and/or (ii) the expression levels of the leukocyte genes selected from a group consisting of TYMS, AK1, F2R, and GNB5 are downregulated or unchanged when comparing the first biological sample (e.g, the first blood sample) to the second biological sample (e.g., the second blood sample).

The present invention may also feature a method for determining the effectiveness of a treatment for severe temporal lobe epilepsy (TLE) in a subject in need thereof. The method may comprise obtaining a first biological sample (e.g., a first blood sample) from the subject, measuring the expression levels of one or more leukocyte genes in the first biological sample (e.g., the first blood sample) from the subject, administering a treatment to the subject, obtaining a second biological sample (e.g., a second blood sample) from the subject, and measuring the expression levels of one or more leukocyte genes in the second biological sample (e.g., the second blood sample) from the subject. In some embodiments, the treatment is effective if (i) the expression levels of the one or more leukocyte genes selected from a group consisting (or consisting essentially) of TYMS, AK1, F2R, and GNB5 are downregulated and/or (ii) the expression levels of NCF2, HMOX1, FCGR2A, PRKCD, CHP1, IFNGR1, RHOB, RAC2, TLR1, TNFRSF1A, LYN, MYD88, and CASP are upregulated when comparing the first biological sample (e.g, the first blood sample) to the second biological sample (e.g., the second blood sample). In some embodiments, the treatment is ineffective if the expression levels of one or more leukocyte genes selected from a group consisting (or consisting essentially) of TYMS, AK1, F2R, and GNB5 are upregulated or unchanged and/or (ii) the expression levels of NCF2, HMOX1, FCGR2A, PRKCD, CHP1, IFNGR1, RHOB, RAC2, TLR1, TNFRSF1A, LYN, MYD88, and CASP are downregulated or unchanged when comparing the first biological sample (e.g, the first blood sample) to the second biological sample (e.g., the second blood sample).

Non-limiting examples of treatments include but are not limited to anti-seizure medication therapy, vagus nerve stimulation, responsive neurostimulation (RNS), stereotactic stem cell or gene therapy administered into the seizure focus (i.e., hippocampus), amygdalohippocampectomy with or without anterior temporal lobectomy and stereotactic laser ablation amygdalohippocampotomy. The present invention is not limited to the aforementioned treatments and may include any appropriate treatments for TLE.

Without wishing to limit the present invention to any theory or mechanism, it is believed that determining the severity of temporal lobe epilepsy (TLE) in a subject may inform the response to medical or surgical treatments and predict their success. Since high and low seizure frequencies exhibit distinct molecular profiles, such as differences in leukocyte gene expression, treatments tailored to these differing pathophysiologies are expected to vary in effectiveness. Therefore, the success of a treatment may depend on whether leukocyte gene expression indicates the presence of high or low seizure frequency TLE.

As used herein, “severe TLE” is characterized by the subject having more than two seizures a month, and “mild TLE” is characterized by the subject having less than two seizures a month.

In some embodiments, the subject is medically refractory. As used herein, medically refractory refers to epilepsy that has failed treatment resulting in continued seizures despite the administration of at least two appropriate anti-seizure medications at appropriate therapeutic drug levels.

In some embodiments, the methods described herein may comprise the expression levels of two or more leukocyte genes in the biological sample (e.g., the blood sample) from the subject. In some embodiments, the methods may comprise measuring the expression levels of five or more leukocyte genes in the biological sample (e.g., the blood sample) from the subject. In some embodiments, the methods may comprise measuring the expression levels of eight or more leukocyte genes in the biological sample (e.g., the blood sample) from the subject. In some embodiments, the methods may comprise measuring the expression levels of ten or more leukocyte genes in the biological sample (e.g., the blood sample) from the subject. In some embodiments, the methods may comprise measuring the expression levels of fifteen or more leukocyte genes in the biological sample (e.g., the blood sample) from the subject. In some embodiments, the methods may comprise measuring the expression levels of seventeen or more leukocyte genes in the biological sample (e.g., the blood sample) from the subject.

In some embodiments, the expression levels of the leukocyte genes are compared to a predetermined threshold. In other embodiments, the expression levels of the leukocyte genes are compared to a healthy control (e.g., a non-epileptic subject). In further embodiments, the expression levels of the leukocyte genes are compared to a sample previously collected from the subject.

In some embodiments, a control subject (e.g., a non-epileptic individual) is expected to exhibit leukocyte gene expression that shows no signs of the temporal lobe epilepsy triad, which includes inflammation, oxidative stress, and dysregulation of excitatory or inhibitory neurotransmitter genes.

The leukocyte genes may be selected from a group comprising Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), Caspase-1 (CASP1), or a combination thereof. Additionally, the leukocyte genes are involved in (a) neuroinflammation, (b) oxidative stress and lipid peroxidation, and (c) glutamate/GABA-mediated excitotoxicity. Thus, other leukocyte genes involved in the aforementioned pathways may be used in accordance with the present invention.

Methods herein may further comprise measuring the levels of leukocyte genes involved in N-methyl-D-aspartate (NMDA)- and Gamma-Aminobutyric Acid (GABA)-mediated epileptogenicity. The leukocyte genes may be selected from a group comprising Thymidylate Synthase (TYMS), Adenylate Kinase 1 (AK1), Coagulation Factor II Thrombin Receptor (F2R), G Protein Subunit Beta 5 (GNB5), or a combination thereof. In some embodiments, the subject is predicted to have a high seizure frequency if the levels of the leukocyte genes are upregulated. In some embodiments, the subject is predicted to have low seizure frequency if the levels of the leukocyte genes are down-upregulated.

### Example

The following is a non-limiting example of the present invention. It is to be understood that said example is not intended to limit the present invention in any way. Equivalents or substitutes are within the scope of the present invention.

The current study was performed to test the hypothesis that systemic leukocyte gene expression differentiates low from high TLE seizure frequency. The results show that low seizure frequency TLE is differentiated from high seizure frequency TLE based on leukocyte gene expression. Low and high seizure frequency TLE are predicted by the respective upregulation and downregulation of specific leukocyte genes involved in canonical pathways of neuroinflammation, oxidative stress and lipid peroxidation, GABA (γ-aminobutyric acid) inhibition, and AMPA (α-amino-3-hydroxy-5-methyl-4-isoxazolepropionic acid) and NMDA (N-Methyl-d-aspartate) receptor signaling. Furthermore, high seizure frequency-TLE is distinguished prognostically from low seizure frequency-TLE by differentially increased specific leukocyte gene expression involved in GABA (γ-aminobutyric acid) inhibition and NMDA (N-Methyl-d-aspartate) receptor signaling. We also explore what appears to be a consistent pattern of processes involving phagosome formation, CREB (CAMP response element-binding protein) signaling and autophagy being suppressed or enhanced in the leukocytes of high and low seizure frequency patients and how these two groups appear to represent two mechanistically different forms of TLE based on leukocyte gene expression.

Patient Characteristics: This study involves sixteen consecutive patients referred for evaluation at the Arizona Comprehensive Epilepsy Program (Tucson, AZ) to characterize seizure phenomenology and determine candidacy for epilepsy surgery. In all patients, the diagnosis of medically intractable epilepsy was made based on seizures being refractory to at least two appropriately prescribed anticonvulsant medications, either in monotherapy or in combination. All sixteen patients underwent MRI brain scanning to eliminate the presence of a space-occupying, structural lesion. In all sixteen patients, long-term video/EEG (electroencephalographic) recording was performed, resulting in the diagnosis of complex partial seizures. For patients in whom long-term noninvasive scalp-EEG failed to localize the ictal temporal lobe seizure focus, long-term invasive EEG recording was performed with either subdural or depth EEG. In all sixteen patients, the baseline seizure frequency was defined as low (<2 seizures/month) or high (>2 seizures/month). In all sixteen patients, a complete history of all prior and current anticonvulsant medication prescriptions was obtained. In some patients, additional noninvasive evaluation with brain PET (positron emission tomography) and neuropsychological testing was performed to assist in seizure focus localization. All non-electrophysiological seizure focus localizing data were evaluated for concordance or dis-concordance with definitive ictal EEG seizure focus localization based on long-term scalp-EEG or, when necessary, invasive ictal EEG recording.

This study was conducted in accordance with the protocols and consent forms approved by the University of Arizona Institutional Review Board.

Leukocyte Gene Expression and Pathway Analysis: In all sixteen patients, whole blood samples were obtained after all seizure focus localizing data confirmed a unilateral ictal temporal lobe seizure focus. Whole blood was stored in PaxGene RNA stabilization fluid (Qiagen, Valencia, CA) at −80° C. The technique for analyzing leukocyte gene expression was performed. Briefly, using the RNeasy lipid tissue mini kit (Qiagen, Valencia, CA), total leukocyte RNA extraction was performed according to manufacturer's directions. The SuperScript III kit (Life Technologies/Thermo Fisher Scientific, Carlsbad, CA) was used to produce first strand cDNA. The High Sensitivity RNA Analysis Kit (Fragment Analyzer; Advanced Analytical Technologies, Ankeny, IA) provided RNA quality assessment. Quant-iT RiboGreen RNA Assay Kit (Molecular Probes; Thermo Fisher Scientific, Carlsbad, CA) determined the concentration of the isolated RNA. The stranded mRNA-Seq Kit (TDS KR0960-v3.15; KapaBiosystems, Wilmington, MA) was used to construct RNA Sequence (RNA-Seq) Libraries. The average fragment size and quality was determined by using the fragment Analyzer (Advanced Analytical Technologies, Ankeny, IA). The library concentration was assessed using the Illumina Universal Adaptor-specific qPCR kit (KapaBiosystems, Wilmington, MA). Sequencing was performed on the HiSeq. 2500 (Illumina, San Diego, CA) with pooled and clustered equimolar samples and by using the Rapid-Run SBS 2×100 bp chemistry (Illumina, San Diego, CA).

Data Analysis: Trimmomatic (USADelLab, Aachen, Germany) was used to trim and quality filter sample data. STAR aligner version 2.5.2b was used to align Fastq files against the GRCh37 reference genome and Htseq-count version 0.6.1 was used to produce gene expression counts. EdgeR's exactTest function was used to calculate differential expression. To eliminate composition biases between library samples, the calcNormFactors function in edgeR was used to normalize gene expression counts using the trimmed mean of M values (TMM), creating a set of scaling factors. All significant differentially expressed genes (FDR<0.05) were analyzed with Ingenuity® Pathway Analysis (IPA®) to identify biological pathways with predictive value for low versus high temporal lobe epilepsy seizure frequency and for functional annotations wherein clustering of genes was significantly upregulated or downregulated (Qiagen, Hilden, Germany). IPA also predicted significant upstream transcriptional regulators. MDS plots were constructed using edgeR's “plotMDS” function, which plots samples on a two-dimensional scatterplot so that distances on the plot approximate the typical log 2 fold changes between samples. Heat maps were made using the R package heatmap version 1.0.12 implemented on the raw read count matrix.

TABLE 1
Patient Clinical Demographics for Temporal Lobe Epilepsy Series

| | | Age | BSF | | Duration | |
|---|---|---|---|---|---|---|
| Subject # | Gender | (yrs) | (sz/mo) | Etiology | (yrs) | Laterality |
| 1 | M | 38 | 3 | Unk | 17 | R |
| 2 | M | 37 | 0.25 | Unk | 35 | L |
| 3 | M | 60 | 0.25 | Unk | 47 | L |
| 4 | M | 26 | 1 | Unk | 4 | R |
| 5 | F | 32 | 0.33 | CVA | 8 | R |
| 6 | M | 16 | 4 | Unk | 10 | R |
| 7 | F | 35 | 1 | Unk | 13 | R |
| 8 | F | 54 | 1 | Ecl | 36 | L |
| 9 | F | 45 | 4 | Abor | 8 | R |
| 10 | M | 46 | 2 | Unk | 43 | L |
| 11 | M | 19 | 60 | TBI | 7 | L |
| 12 | F | 62 | 2 | Inf | 61 | L |
| 13 | M | 32 | 2 | Unk | 25 | L |
| 14 | M | 26 | 1 | Unk | 19 | L |
| 15 | M | 45 | 4 | Unk | 37 | L |
| 16 | F | 58 | 2 | Unk | 37 | R |

Etiology = Etiology of epilepsy; TBI = traumatic brain injury; Unk = unknown; CVA = stroke; Abor = abortion; Inf = infection; Ecl = eclampsia; Duration = duration of epilepsy; Laterality = laterality of ictal temporal lobe seizure focus, L = left, R = right; BSF = baseline seizure frequency, sz/mo = seizures per month.

TABLE 2
Antiepileptic Medication Use of
Patients in Temporal Lobe Epilepsy Series.

| | USE | LOW | HIGH | |
|---|---|---|---|---|
| | (YES/ | SEIZURE | SEIZURE | P- |
| MEDICATION | NO) | FREQUENCY | FREQUENCY | VALUE# |
| CARBAMAZEPINE | Yes | 8 | 1 | 0.26 |
| | No | 4 | 3 | |
| PHENYTOIN | Yes | 6 | 3 | 0.58 |
| | No | 6 | 1 | |
| VALPROIC ACID | Yes | 4 | 2 | 1.00 |
| | No | 8 | 2 | |
| OXCARBAZEPINE | Yes | 3 | 1 | 1.00 |
| | No | 9 | 3 | |
| GABAPENTIN | Yes | 2 | 0 | 1.00 |
| | No | 10 | 4 | |
| TOPIRAMATE | Yes | 3 | 1 | 1.00 |
| | No | 9 | 3 | |
| PHENOBARBITAL | Yes | 3 | 1 | 1.00 |
| | No | 9 | 3 | |
| ZONISAMIDE | Yes | 2 | 0 | 1.00 |
| | No | 10 | 4 | |
| LEVETIRACETAM | Yes | 8 | 2 | 1.00 |
| | No | 4 | 2 | |
| VIGABATRIN | Yes | 0 | 1 | 0.25 |
| | No | 12 | 3 | |
| LACOSAMIDE | Yes | 1 | 1 | 1.00 |
| | No | 11 | 3 | |
| LAMOTRIGINE | Yes | 7 | 2 | 1.00 |
| | No | 5 | 2 | |
| OTHER* | Yes | 10 | 3 | 1.00 |
| | No | 2 | 1 | |

*Other = Lorazepam, Zomig, Clonazepam, Clobazam, Primidone, Fycompa, Temazepam, Mysoline, Diazepam;
#Fisher exact test.
Reproduced, in part, with permission of Sprissler et al (Sprissler R, Bina R, Kasoff W, et al. Leukocyte Expression Profiles Reveal Gene Sets With Prognostic Value for Seizure-Free Outcome Following Stereotactic Laser Amygdalohippocampotomy. Sci Rep. 2019; 9(1): 1086. https://doi.org/10.1038/s41598-018-37763-5).

TABLE 3
Demographic and Seizure Focus Localization Data of Temporal Lobe Epilepsy Series.

| | | HIGH SEIZURE | LOW SEIZURE | |
|---|---|---|---|---|
| DEMOGRAPHIC | FACTORS | FREQUENCY | FREQUENCY | P-VALUE* |
| GENDER | Male | 4 | 6 | 1.00 |
| | Female | 1 | 5 | |
| AGE (YEARS) | Mean (SEM) | 32.6 (6.3) | 42.6 (4.2) | 0.31# |
| DURATION | Mean (SEM) | 15.8 (5.6) | 29.8 (5.3) | 0.17# |
| (YEARS) | | | | |
| ETHNICITY | Caucasian | 2 | 7 | 0.60 |
| | Hispanic/Other | 3 | 4 | |
| MRI BRAIN | MTS | 3 | 8 | 1.00 |
| | Normal/Other | 2 | 3 | |
| PET SCAN | Concordant | 2 | 9 | 0.18 |
| | Dis-concordant | 2 | 1 | |
| ICTAL SCALP | Concordant | 4 | 11 | 0.31 |
| EEG | Dis-concordant | 1 | 0 | |
| INTRACRANIAL | Localizing | 3 | 3 | 1.00 |
| EEG | Non-localizing | 0 | 0 | |
| NEUROPSYCH | Concordant | 3 | 3 | 0.55 |
| | Dis-concordant | 1 | 5 | |
| LATERALITY | Left | 2 | 6 | 1.00 |
| | Right | 3 | 5 | |

*Fisher exact test;
#Mann-Whitney U test. High & Low seizure frequency: > or <2/month,
Concordant/Dis-concordant = Concordance or dis-concordance with temporal lobe ictal EEG seizure focus localization,
MTS = medial temporal sclerosis,
Duration = duration of epilepsy,
Neuropsych = neuropsychological testing;
Laterality = temporal lobe seizure focus lateralization (left versus right).
Reproduced, in part, by permission of Sprissler et al.

TABLE 4
Leukocyte Differentially Expressed Genes (DEGs) comparing
Low and High Temporal Lobe Epilepsy Seizure Frequency Patients.

| | | | |
|---|---|---|---|
| Genes | Fold Change (FC) | FDR* | p-value |

Upregulated in LOW compared to HIGH seizure frequency TLE

| | | | |
|---|---|---|---|
| NCF2 | 4.044962 | 0.002835 | 4.83 × 10⁻⁶ |
| HMOX1 | 3.912648 | 0.001222 | 1.04 × 10⁻⁶ |
| RHOB | 3.370525 | 0.023331 | 0.000122 |
| FCGR2A | 3.355795 | 0.012951 | 4.92 × 10⁻⁵ |
| PRKCD | 3.068704 | 0.010221 | 3.32 × 10⁻⁵ |
| RAC2 | 3.041648 | 0.034416 | 0.00029 |
| TLR1 | 2.869047 | 0.035484 | 0.000348 |
| CHP1 | 2.769877 | 0.008084 | 2.15 × 10⁻⁵ |
| TNFRSF1A | 2.732864 | 0.023331 | 0.00013 |
| IFNGR1 | 2.732414 | 0.016528 | 6.99 × 10⁻⁵ |
| LYN | 2.525921 | 0.031583 | 0.00023 |
| MYD88 | 2.520108 | 0.043643 | 0.000496 |
| CASP1 | 2.443742 | 0.035484 | 0.00033 |

Upregulated in HIGH compared to LOW seizure frequency TLE

| | | | |
|---|---|---|---|
| TYMS | 444.9515 | 0.000136 | 9.58 × 10⁻⁹ |
| AK1 | 59.63923 | 0.009746 | 2.93 × 10⁻⁵ |
| F2R | 51.47103 | 0.015462 | 6.43 × 10⁻⁵ |
| GNB5 | 22.58811 | 0.032269 | 2.62 × 10⁻⁴ |

FC indicates fold change of leukocyte gene upregulation in low versus high seizure frequency TLE.
*FDR = False Discovery Rate. (p < 0.001, FC > 2.0, FDR < 0.05)

TABLE 5
Ingenuity ® Pathway Analysis (IPAR) involving the DEGs with significant
activation of biological pathways when comparing low to high seizure frequency groups

| | | | |
|---|---|---|---|
| Biological Pathways | Genes | z-score | p-value |

Upregulated in LOW compared to HIGH seizure frequency TLE

| | | | |
|---|---|---|---|
| Neuroinflammation Signaling | CASP1, CD86, CHP1, CX3CR1, | 2.887 | 8.32 × 10⁻⁷ |
| Pathway | FZD1, HMOX1, IFNGR1, MYD88, | | |
| | NCF2, SOD2, TLR1, TNFRSF1A | | |
| Fcγ Receptor-mediated | FCGR2A, FCGR3A/FCGR3B, | 2.646 | 2.0 × 10⁻⁶ |
| Phagocytosis in Macrophages | HMOX1, LYN, PRKCD, PTEN, | | |
| and Monocytes | RAC2 | | |
| Production of Nitric Oxide and | CAT, IFINGR1, LYZ, NCF2, | 2.121 | 4.9 × 10⁻⁶ |
| Reactive Oxygen Species in | PRKCD, RAC2, RHOB, SIRPA, | | |
| Macrophages | TNFRSF1A | | |
| Phospholipase C Signaling | ARHGEF11, CHP1, FCGR2A, | 2.449 | 5.6 × 10⁻⁵ |
| | GNB5, HMOX1, LYN, PRKCD, | | |
| | RAC2, RHOB | | |
| Role of Pattern Recognition | C3AR1, C5AR1, CASP1, MYD88, | 2.449 | 7.9 × 10⁻⁵ |
| Receptors in Recognition of | NLRC4, PRKCD, TLR1 | | |
| Bacteria and Viruses | | | |

Upregulated in HIGH compared to LOW seizure frequency TLE

| | | |
|---|---|---|
| Pyrimidine Deoxyribonucleotides | AK1, TYMS | 4.17 × 10⁻⁴ |
| De Novo Biosynthesis I* | | |
| AMPK Signaling* | AK1, GNB5, PPM1K | 4.47 × 10⁻³ |
| Thrombin Signaling* | F2R, GNB5 | 3.39 × 10⁻² |

Canonical Pathways with statistically significant p-values (p < 0.0001 and z-score > 2.000)
*indicates z-score unable to be calculated (NaN) for pathway by Ingenuity ® Pathway Analysis (IPA ®)

TABLE 6
Top most significantly altered IPA canonical pathways

| | p-value | z-score |
|---|---|---|
| 1. Phagosome Formation | 1.27 × 10⁻⁸ | −4.03 |
| 2. Neuroinflammation Signaling Pathway | 1.02 × 10⁻⁷ | −2.89 |
| 3. CREB Signaling in Neurons | 1.29 × 10⁻⁶ | −2.84 |
| 4. Fcγ Receptor-mediated Phagocytosis | 2.15 × 10⁻⁶ | −2.65 |
| in Macrophages and Monocytes | | |
| 5. Autophagy | 6.17 × 10⁻⁵ | −2.65 |
| 6. Production of Nitric Oxide and Reactive | 3.47 × 10⁻⁵ | −2.12 |
| Oxygen Species in Macrophages | | |
| 7. fMLP Signaling in Neutrophils | 1.95 × 10⁻⁵ | −2.00 |
| 8. Role of Pattern Recognition Receptors in | 5.89 × 10⁻⁵ | −2.45 |
| Recognition of Bacteria and Viruses | | |
| 9. CXCR4 Signaling | 9.12 × 10⁻⁵ | −2.24 |
| 10. TREM1 Signaling | 1.26 × 10⁻⁴ | −2.24 |
| 11. IL-8 Signaling | 1.10 × 10⁻⁴ | −2.45 |

Patient Characteristics: A total of sixteen patients participated in this study (Table 1). All sixteen patients met the Task Force of the ILAE (International League Against Epilepsy) Commission on Therapeutic Strategies definition of drug-resistant epilepsy. All sixteen patients were determined by the University of Arizona Comprehensive Epilepsy Program to have intractable complex partial seizures originating from a single temporal lobe. Among the sixteen patients studied, there were ten males and six females with a mean age of 39.4 years (range: 16 to 62 years, standard error of mean: 3.6 years). The median baseline seizure frequency was 2.0 seizures per month (range: 0.25 to 60 seizures per month). There were five patients with high (>2 seizures/month) and eleven patients with low (<2 seizures/month) seizure frequency. The mean duration of temporal lobe epilepsy was 25.4 years (range: 4-61 years, standard error of mean: 4.3 years). There were eight patients with right- and eight patients with left-sided temporal lobe ictal seizure foci. The etiology of intractable temporal lobe epilepsy included stroke (n=1), eclampsia (n=1), traumatic brain injury (n=1), infection (n=1), abortion (n=1), and unknown causes (n=11) (Table 1). There were no statistically significant differences between the patients with high and low seizure frequency based on anticonvulsant medications taken (Table 2). There were no significant differences between the high seizure frequency (HSF) and low seizure frequency (LSF) groups on the basis of gender, age, ethnicity, duration of epilepsy, lateralization of temporal lobe epilepsy seizure foci, MRI brain evidence of medial temporal sclerosis, and concordance or dis-concordance of PET scan, neuropsychological testing, and ictal scalp or intracranial EEG data with definitive seizure focus localization (Table 3).

Leukocyte Gene Differential Expression Analysis: Leukocyte differential gene expression analysis was performed comparing the high (n=5) and low (n=11) seizure frequency groups. Based on a threshold of 2-fold change (p<0.001, FC>2.0, FDR<0.05) and the involvement in at least two biological pathways in both Reactome and Ingenuity® Pathway Analysis (IPA®), 13 differentially expressed genes (DEGs) were identified which were all overexpressed in the low when compared to the high seizure frequency groups (Table 4). Using the same filter criteria, four differentially expressed genes (DEGs) were identified which were all overexpressed in the high when compared to the low seizure frequency groups (Table 4). It should be noted that there are no control leukocytes to establish baseline gene expression levels. Thus, it cannot be inferred whether particular genes are up- or down-regulated in leukocytes from LSF or HSF patients. Instead, relative levels of expression in comparison between the two seizure frequency groups were inferred. The same is true for inferring whether particular pathways are activated or deactivated.

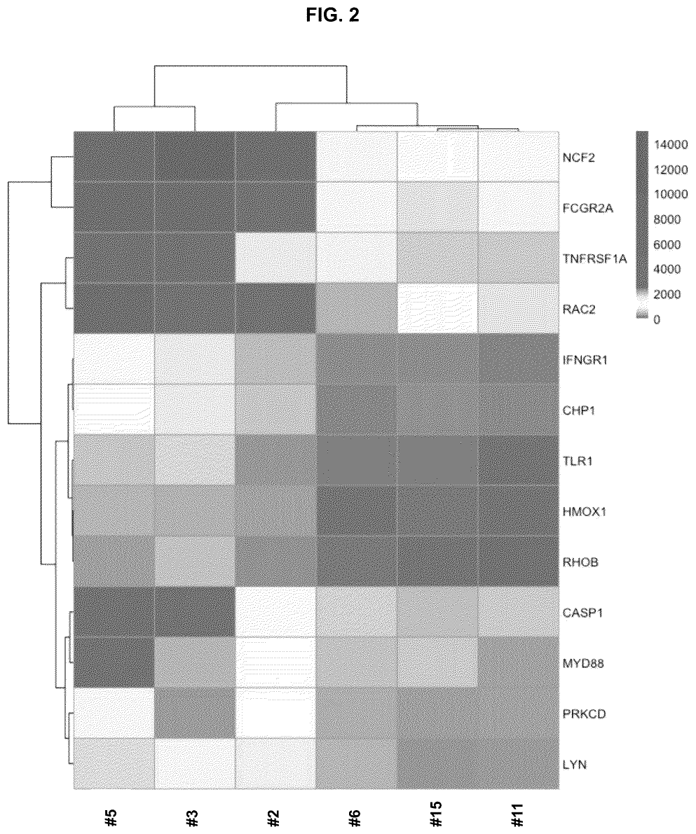

Multidimensional Scaling Plot (MDS) and Heatmap Analysis: To further illustrate the expression profile differences between the low and high seizure frequency groups, an MDS plot was generated using the three patients with the lowest seizure frequency and the three patients with the highest seizure frequency. This plot generated a clear division between these two cohorts indicating variable inter-group expression profiles (FIG. 1). Again, using the three lowest and highest seizure frequency patients from the cohort, a heatmap was generated to assess the overall expression level of each identified DEG and classify as either a high or low expressing gene by the actual quantity of transcripts within the sample (FIG. 2).

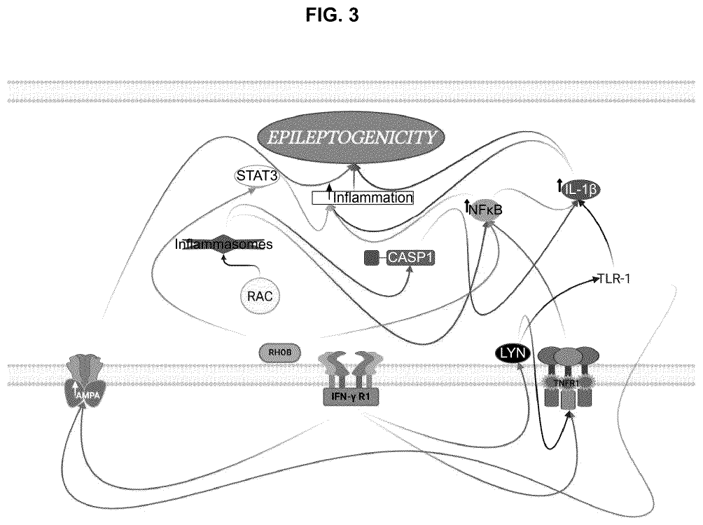

Pathway Analysis: Pathway analysis was performed on the above DEGs to detect significantly activated biological pathways in the low compared to the high seizure frequency group (p<0.0001, z-score >2.000) (Table 5). The results show that low human TLE seizure frequency is differentiated from high seizure frequency by the respective upregulation versus downregulation of specific leukocyte DEGs involving neuroinflammation, oxidative stress and lipid peroxidation, GABA (γ-aminobutyric acid) inhibition, and AMPA (α-amino-3-hydroxy-5-methyl-4-isoxazolepropionic acid) and NMDA (N-Methyl-d-aspartate) receptor signaling (FIGS. 3-5).

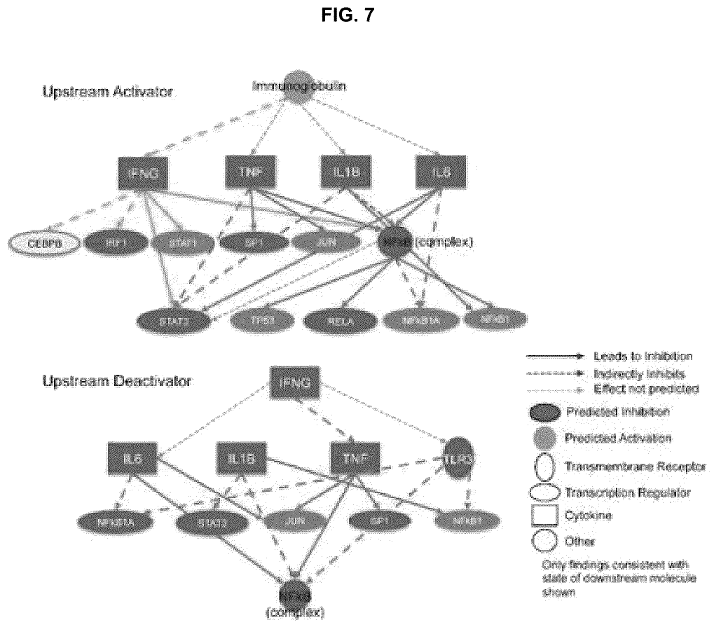

This same analysis was performed on the high compared to low DEGs to identify significantly activated biological pathways in the high compared to deactivated biological pathways in the low seizure frequency group (Table 5). These results indicate that high human TLE seizure frequency is differentiated from low seizure frequency by the respective upregulation versus downregulation of specific leukocyte DEGs involving NMDA receptor facilitation and GABAergic inhibition (FIG. 6). Additionally, the top altered canonical pathways were then inferred from pathway enrichment analysis. These include several related functions involved in phagosome formation, neuroinflammation signaling, CREB signaling, and autophagy showing a consistent pattern of suppression or enhancement in the leukocytes of HSF or LSF patients, respectively (Table 6). Predicted upstream transcriptional regulators can identify drivers of the differential expression in TLE. Of the biological molecules predicted to be inhibitory, IFNG was the regulator with the lowest p-value (4.94×10⁻¹⁹) and the most negative z-score (−4.71) (FIG. 7). Immunoglobulin was the top predicted upstream activator with a p-value of 3.10×10⁻¹² and a z-score of 2.70 (data not shown).

High and low seizure frequency TLE patients appear to represent two mechanistically different forms of temporal lobe epilepsy based on leukocyte gene expression. Low versus high seizure frequency TLE is predicted by the respective overexpression and underexpression of specific systemic leukocyte DEGs involving neuroinflammation, oxidative stress and lipid peroxidation, GABA (γ-aminobutyric acid) inhibition, and AMPA (γ-amino-3-hydroxy-5-methyl-4-isoxazolepropionic acid) and NMDA (N-Methyl-d-aspartate) receptor signaling. In addition, high seizure frequency-temporal lobe epilepsy is distinguished prognostically from low seizure frequency-temporal lobe epilepsy by differentially increased specific leukocyte gene expression involved in GABA (γ-aminobutyric acid) inhibition and NMDA (N-Methyl-d-aspartate) receptor signaling. Additionally, there appears to be a larger scale suppression or enhancement of phagosome formation, CREB signaling, and autophagy between the low and high seizure frequency cohorts that may also inform the mechanistic differences in these patients with TLE.

### Embodiments

The following embodiments are intended to be illustrative only and not to be limiting in any way.

Embodiment 1: A method of predicting the severity of human temporal lobe epilepsy (TLE) in a subject in need thereof, the method comprising: a) obtaining a biological sample from the subject; and b) measuring expression levels of one or more leukocyte genes in the biological sample from the subject; wherein the subject is predicted to have severe TLE if the expression levels of the one or more leukocyte genes are down-regulated, or the subject is predicted to have mild TLE if the expression levels of the one or more leukocyte genes are upregulated.

Embodiment 2: The method of embodiment 1, wherein the subject is medically refractory.

Embodiment 3: The method of embodiment 1 or embodiment 2, wherein the biological sample comprises a blood sample, a brain sample, spinal fluid, a cheek swab, or a skin sample.

Embodiment 4: The method of any one of embodiments 1-3, wherein severe TLE is characterized by the subject having more than two seizures a month, and wherein mild TLE is characterized by the subject having less than two seizures a month.

Embodiment 5: The method of any one of embodiments 1-4, wherein the expression level of five or more leukocyte genes is measured.

Embodiment 6: The method of any one of embodiments 1-4, wherein the expression level of ten or more leukocyte genes is measured.

Embodiment 7: The method of any one of embodiments 1-6, wherein the one or more leukocyte genes are selected from a group comprising one or a combination of Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), and Caspase-1 (CASP1).

Embodiment 8: The method of any one of embodiments 1-7 further comprising measuring the expression levels of one or more leukocyte genes selected from a group comprising one or a combination of Thymidylate Synthase (TYMS), Adenylate Kinase 1 (AK1), Coagulation Factor II Thrombin Receptor (F2R), G Protein Subunit Beta 5 (GNB5).

Embodiment 9: The method of embodiment 8, wherein the subject is predicted to have a high seizure frequency if the expression levels of the leukocyte genes are upregulated or the subject is predicted to have low seizure frequency if the expression levels of the leukocyte genes are down-upregulated.

Embodiment 10: A method of predicting the severity of human temporal lobe epilepsy (TLE) in a subject in need thereof, the method comprising: a) obtaining a blood sample from the subject; and b) measuring the expression levels of ten or more leukocyte genes in the blood sample from the subject; wherein the subject is predicted to have severe TLE if (i) the expression levels of the leukocyte genes selected from a group consisting of Thymidylate Synthase (TYMS), Adenylate Kinase 1 (AK1), Coagulation Factor II Thrombin Receptor (F2R), and G Protein Subunit Beta 5 (GNB5) are upregulated and/or (ii) the expression level of the leukocyte genes selected from a group consisting of Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), and Caspase-1 (CASP1) are down-regulated; and wherein the subject is predicted to have mild TLE if (i) the expression levels of the leukocyte genes selected from a group consisting of NCF2, HMOX1, FCGR2A, PRKCD, CHP1, IFNGR1, RHOB, RAC2, TLR1, TNFRSF1A, LYN, MYD88, and CASP are upregulated and/or (ii) the expression levels of the leukocyte genes selected from a group consisting of TYMS, AK1, F2R, and GNB5 are downregulated.

Embodiment 11: The method of embodiment 10, wherein the subject is medically refractory.

Embodiment 12: The method of embodiment 10 or embodiment 11, wherein severe TLE is characterized by the subject having more than two seizures a month, and wherein mild TLE is characterized by the subject having less than two seizures a month.

Embodiment 13: The method of any one of embodiments 10-12, wherein the expression level of fifteen or more leukocyte genes is measured.

Embodiment 14: A method of treating a subject with temporal lobe epilepsy (TLE), the method comprising: a) determining the severity of the TLE in a subject in need thereof by; ii) obtaining a biological sample from the subject; and ii) measuring the expression levels of one or more leukocyte genes in the biological sample from the subject; wherein if the expression levels of the one or more leukocyte genes are down-regulated the subject has severe TLE, and if the expression levels of the one or more leukocyte genes are upregulated the subject has mild TLE, and b) administering a treatment to the subject based on whether the subject has severe TLE or mild TLE.

Embodiment 15: The method of embodiment 14, wherein the subject is medically refractory.

Embodiment 16: The method of embodiment 14 or embodiment 15, wherein the biological sample comprises a blood sample, a brain sample, spinal fluid, a cheek swab, or a skin sample.

Embodiment 17: The method of any one of embodiments 14-16, wherein severe TLE is characterized by the subject having more than two seizures a month, and wherein mild TLE is characterized by the subject having less than two seizures a month.

Embodiment 18: The method of any one of embodiments 14-17, wherein the expression level of five or more leukocyte genes are measured.

Embodiment 19: The method of any one of embodiments 14-17, wherein the expression level of ten or more leukocyte genes is measured.

Embodiment 20: The method of any one of embodiments 14-19, wherein the one or more leukocyte genes are selected from a group comprising one or a combination of Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), and Caspase-1 (CASP1).

Embodiment 21: The method of any one of embodiments 14-20 further comprising measuring the expression levels of one or more leukocyte genes selected from a group comprising one or a combination of Thymidylate Synthase (TYMS), Adenylate Kinase 1 (AK1), Coagulation Factor II Thrombin Receptor (F2R), G Protein Subunit Beta 5 (GNB5).

Embodiment 22: The method of embodiment 21, wherein the subject is predicted to have a high seizure frequency if the levels of the leukocyte genes are upregulated or the subject is predicted to have low seizure frequency if the levels of the leukocyte genes are down-upregulated.

Embodiment 23: A method of treating a subject with temporal lobe epilepsy (TLE), the method comprising: a) determining the severity of the TLE in a subject in need thereof by; i) obtaining a blood sample from the subject; and ii) measuring the expression levels of ten or more leukocyte genes in the blood sample from the subject; wherein the subject is predicted to have severe TLE if (i) the expression levels of the leukocyte genes selected from a group consisting of Thymidylate Synthase (TYMS), Adenylate Kinase 1 (AK1), Coagulation Factor II Thrombin Receptor (F2R), and G Protein Subunit Beta 5 (GNB5) are upregulated and/or (ii) the expression level of the leukocyte genes selected from a group consisting of Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), and Caspase-1 (CASP1) are down-regulated; and wherein the subject is predicted to have mild TLE if (i) the expression level of the leukocyte genes selected from a group consisting of NCF2, HMOX1, FCGR2A, PRKCD, CHP1, IFNGR1, RHOB, RAC2, TLR1, TNFRSF1A, LYN, MYD88, and CASP are upregulated and/or (ii) the expression levels of the leukocyte genes selected from a group consisting of TYMS, AK1, F2R, and GNB5 are downregulated; and b) administering a treatment to the subject based on whether the subject has severe TLE or mild TLE.

Embodiment 24: The method of embodiment 23, wherein the subject is medically refractory.

Embodiment 25: The method of embodiment 23 or embodiment 24, wherein severe TLE is characterized by the subject having more than two seizures a month, and wherein mild TLE is characterized by the subject having less than two seizures a month.

Embodiment 26: The method of any one of embodiments 23-25, wherein the expression level of fifteen or more leukocyte genes is measured.

Embodiment 27: A method for determining the effectiveness of a treatment for human temporal lobe epilepsy (TLE) in a subject in need thereof, the method comprising: a) obtaining a first blood sample from the subject; b) measuring the expression levels of one or more leukocyte genes in the first blood sample from the subject; c) administering a treatment to the subject; d) obtaining a second blood sample from the subject; and e) measuring the expression levels of one or more leukocyte genes in the second blood sample from the subject; wherein the treatment is effective if the levels of the one or more leukocyte genes are upregulated, and the treatment is ineffective if the levels of the one or more leukocyte genes are down-regulated.

Embodiment 28: The method of embodiment 27, wherein the one or more leukocyte genes are selected from a group comprising Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), Caspase-1 (CASP1), or a combination thereof.

Embodiment 29: The method of embodiment 27 or embodiment 28 further comprising measuring the expression levels leukocyte genes involved in N-methyl-D-aspartate (NMDA)- and Gamma-Aminobutyric Acid (GABA)-mediated epileptogenicity; wherein the leukocyte genes are selected from a group comprising Thymidylate Synthase (TYMS), Adenylate Kinase 1 (AK1), Coagulation Factor II Thrombin Receptor (F2R), G Protein Subunit Beta 5 (GNB5), or a combination thereof.

Embodiment 30: The method of embodiment 29, wherein the treatment is effective if the levels of the leukocyte genes are down-upregulated and wherein the treatment is ineffective if the levels of the leukocyte genes are upregulated.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only, and the claims are not limited by the dimensions of the figures. In some embodiments, descriptions of the inventions described herein using the phrase “comprising” includes embodiments that could be described as “consisting essentially of” or “consisting of,” and as such, the written description requirement for claiming one or more embodiments of the present invention using the phrase “consisting essentially of” or “consisting of” is met.

What is claimed is:

1. A method of predicting the severity of human temporal lobe epilepsy (TLE) in a subject in need thereof, the method comprising:
a) obtaining a biological sample from the subject; and
b) measuring the expression levels of one or more leukocyte genes in the biological sample from the subject;
wherein the subject is predicted to have severe TLE if the levels of the one or more leukocyte genes are down-regulated, or the subject is predicted to have mild TLE if the levels of the one or more leukocyte genes are upregulated.

2. The method of claim 1, wherein the subject is medically refractory.

3. The method of claim 1, wherein the biological sample comprises a blood sample, a brain sample, spinal fluid, a cheek swab, or a skin sample.

4. The method of claim 1, wherein severe TLE is characterized by the subject having more than two seizures a month, and wherein mild TLE is characterized by the subject having less than two seizures a month.

5. The method of claim 1, wherein the expression level of five or more leukocyte genes is measured.

6. The method of claim 1, wherein the expression level of ten or more leukocyte genes is measured.

7. The method of claim 1, wherein the expression level of fifteen or more leukocyte genes is measured.

8. The method of claim 1, wherein the one or more leukocyte genes are selected from a group comprising one or a combination of Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), and Caspase-1 (CASP1).

9. The method of claim 1 further comprising measuring the levels of one or more leukocyte genes selected from a group comprising one or a combination of Thymidylate Synthase (TYMS), Adenylate Kinase 1 (AK1), Coagulation Factor II Thrombin Receptor (F2R), G Protein Subunit Beta 5 (GNB5).

10. The method of claim 9, wherein the subject is predicted to have a high seizure frequency if the levels of the leukocyte genes are upregulated or the subject is predicted to have low seizure frequency if the levels of the leukocyte genes are down-upregulated.

11. A method of predicting the severity of human temporal lobe epilepsy (TLE) in a subject in need thereof, the method comprising:
a) obtaining a biological sample from the subject; and
b) measuring the expression levels of ten or more leukocyte genes in the biological sample from the subject;
wherein the subject is predicted to have severe TLE if (i) the expression levels of the leukocyte genes selected from a group consisting of Thymidylate Synthase (TYMS), Adenylate Kinase 1 (AK1), Coagulation Factor II Thrombin Receptor (F2R), and G Protein Subunit Beta 5 (GNB5) are upregulated and/or (ii) the expression level of the leukocyte genes selected from a group consisting of Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), and Caspase-1 (CASP1) are down-regulated; and
wherein the subject is predicted to have mild TLE if (i) the expression level of the leukocyte genes selected from a group consisting of NCF2, HMOX1, FCGR2A, PRKCD, CHP1, IFNGR1, RHOB, RAC2, TLR1, TNFRSF1A, LYN, MYD88, and CASP are upregulated and/or (ii) the expression levels of the leukocyte genes selected from a group consisting of TYMS, AK1, F2R, and GNB5 are downregulated.

12. The method of claim 11, wherein the subject is medically refractory.

13. The method of claim 11, wherein the biological sample comprises a blood sample, a brain sample, spinal fluid, a cheek swab, or a skin sample.

14. The method of claim 11, wherein severe TLE is characterized by the subject having more than two seizures a month, and wherein mild TLE is characterized by the subject having less than two seizures a month.

15. The method of claim 11, wherein the expression level of fifteen or more leukocyte genes is measured.

16. A method of treating a subject with temporal lobe epilepsy (TLE), the method comprising:
a) determining the severity of the TLE in a subject in need thereof by;
i. obtaining a biological sample from the subject; and
ii. measuring the expression levels of ten or more leukocyte genes in the biological sample from the subject;
wherein the subject is predicted to have severe TLE if (i) the expression levels of the leukocyte genes selected from a group consisting of Thymidylate Synthase (TYMS), Adenylate Kinase 1 (AK1), Coagulation Factor II Thrombin Receptor (F2R), and G Protein Subunit Beta 5 (GNB5) are upregulated and/or (ii) the expression level of the leukocyte genes selected from a group consisting of Neutrophil Cytosolic Factor 2 (NCF2), Heme Oxygenase (HMOX), Ras Homolog Family Member B (RHOB), Fc Gamma Receptor IIa (FCGR2A), Protein Kinase C Delta (PRKCD), Rac Family Small GTPase 2 (RAC2), Toll Like Receptor 1 (TLR1), Calcineurin Like EF-Hand Protein 1 (CHP1), Tumor Necrosis Factor Receptor Superfamily Member 1A (TNFRSF1A), Interferon Gamma Receptor 1 (IFNGR1), LYN, Myeloid Differentiation Primary Response 88 (MYD88), and Caspase-1 (CASP1) are down-regulated; and
wherein the subject is predicted to have mild TLE if (i) the expression level of the leukocyte genes selected from a group consisting of NCF2, HMOX1, FCGR2A, PRKCD, CHP1, IFNGR1, RHOB, RAC2, TLR1, TNFRSF1A, LYN, MYD88, and CASP are upregulated and/or (ii) the expression levels of the leukocyte genes selected from a group consisting of TYMS, AK1, F2R, and GNB5 are downregulated; and
b) administering a treatment to the subject based on whether the subject has severe TLE or mild TLE.

17. The method of claim 16, wherein the subject is medically refractory.

18. The method of claim 16, wherein the biological sample comprises a blood sample, a brain sample, spinal fluid, a cheek swab, or a skin sample.

19. The method of claim 16, wherein severe TLE is characterized by the subject having more than two seizures a month, and wherein mild TLE is characterized by the subject having less than two seizures a month.

20. The method of claim 16, wherein the expression level of fifteen or more leukocyte genes is measured.
